(19) United States
(12) Patent Application Publication
(10) Pub. No.: US 2011/0020896 A1
(43) Pub. Date: Jan. 27, 2011

(54) MUTANT DNA POLYMERASES AND THEIR GENES

(76) Inventors: Jung Hyun Lee, Gyeonggi-do (KR); Sung Gyun Kang, Gyeonggi-do (KR); Sang Jin Kim, Gyeonggi-do (KR); Kae Kyoung Kwon, Gyeonggi-do (KR); Hyun Sook Lee, Gyeonggi-do (KR); Yun Jae Kim, Gyeonggi-do (KR); Seung Seob Bae, Gyeonggi-do (KR); Jae Kyu Lim, Gyeonggi-do (KR); Jung Ho Jeon, Gyeonggi-do (KR); Yo Na Cho, Gyeonggi-do (KR); Suk Tae Kwon, Gyeonggi-do (KR)

Correspondence Address: CLARK & ELBING LLP, 101 FEDERAL STREET, BOSTON, MA, 02110, US

(21) Appl. No.: 12/444,015

(22) PCT Filed: Oct. 2, 2007
(86) PCT No.: PCT/KR07/04832

§ 371 Date: Oct. 12, 2010

(30) Foreign Application Priority Data
Oct. 2, 2006 (KR) 10-2006-0097450

(51) Int. Cl. C12N 9/12; C07H 21/00; C12N 15/63; C12N 5/10

(52) U.S. Cl. 435/194; 536/23.2; 435/320.1; 435/325

(57) ABSTRACT

The present invention relates to mutant DNA polymerases, their genes and their uses. More specifically, the present invention relates to mutant DNA polymerases which are originally isolated from Thermococcus sp NA1. strain and produced by site-specific mutagenesis, their amino acid sequences, genes encoding said mutant DNA polymerases, their nucleic acids and PCR methods by using thereof. As mutant DNA polymerases according to the present invention have decreased proofreading activity and changed function of inosine sensing effectively compared to wild type DNA polymerase, PCR using primers with specific nucleic acids has made rapid progress. Therefore, the present invention is broadly applicable for PCR in various molecular genetic technologies.

### TECHNICAL FIELD

The present invention relates to mutant DNA polymerases, their genes and their uses. More specifically, the present invention relates to mutant DNA polymerases which is originally isolated from Thermococcus sp. strain and produced by site-specific mutagenesis, their amino acid sequences, genes encoding said mutant DNA polymerases and PCR methods using thereof.

### BACKGROUND ART

The recent advance of genomic research has produced vast amounts of sequence information. With a generally applicable combination of conventional genetic engineering and genomic research techniques, the genome sequences of some hyperthermophilic microorganisms are of considerable biotechnological interest due to heat-stable enzymes, and many extremely thermostable enzymes are being developed for biotechnological purposes.

PCR, which uses the thermostable DNA polymerase, is one of the most important contributions to protein and genetic research and is currently used in a broad array of biological applications. More than 50 DNA polymerase genes have been cloned from various organisms, including thermophiles and archaeas. Recently, family B DNA polymerases from hyperthermophilic archaea, Pyrococcus and Thermococcus, have been widely used since they have higher fidelity in PCR based on their proof reading activity than Taq polymerase commonly used. However, the improvement of the high fidelity enzyme has been on demand due to lower DNA elongation ability.

The present inventors isolated a new hyperthermophilic strain from a deep-sea hydrothermal vent area at the PACMANUS field. It was identified as a member of Thermococcus based on 16S rDNA sequence analysis, and the whole genome sequencing is currently in process to search for many extremely thermostable enzymes. The analysis of the genome information displayed that the strain possessed a family B type DNA polymerase. The present inventors cloned the gene corresponding to the DNA polymerase and expressed in E. coli. In addition, the recombinant enzyme was purified and its enzymatic characteristics were examined. Therefore, the present inventors applied for a patent on the DNA polymerase having high DNA elongation and high fidelity ability (Korean Patent No. 2005-0094644).

Due to strong exonuclease activity and inosine sensing, high fidelity DNA polymerases aren't suitable for PCR using primers with inosine. Accordingly, the present inventors need to develop a DNA polymerase which is suitable for PCR reaction using primer with inosine from the wild type TNA1_pol DNA polymerase of Korean Patent No. 2005-0094644.

Accordingly, as a result of continuous efforts, the present inventors have introduced site-specific mutagenesis at hyperthermophilic DNA polymerases isolated from Thermococcus sp. strain and selected mutant DNA polymerases with a changed exonuclease activity and inosine sensing ability. The identified mutant DNA polymerases are useful for PCR using primer with inosine. Thereby, the present invention has been accomplished.

### DISCLOSURE

### Technical Problem

It is an object of the present invention to provide mutant DNA polymerases and their genes.

### Technical Solution

The present invention provides mutant DNA polymerases produced by site-specific mutagenesis on exonuclease active site and inosine sensing region from the wild type TNA1_pol DNA polymerase, Korean Patent No. 2005-0094644, which is isolated from Thermococcus sp. strain.

Preferably, said exonuclease active site can be one or more motifs selected from the group consisting of ExoI motif, ExoII motif and ExoIII motif.

Also, the present invention provides mutant DNA polymerases produced by one or more mutagenesis simultaneously on inosine sensing domain.

### DESCRIPTION OF DRAWINGS

FIG. 1 shows the results of SDS-PAGE analysis of mutant DNA polymerases.

M: a standard sample, W: wild type, 1: mutant I, 2: mutant II, 3: mutant III

FIG. 2 shows the results of PCR analysis using mutant DNA polymerases, respectively.

M: a standard sample, W: wild type, 1: mutant I, 2: mutant II, 3: mutant III

FIG. 3 shows the results of PCR analysis using mutant DNA polymerases and primer with inosine, respectively.

M: a standard sample, W: wild type, 1: mutant I, 2: mutant II, 3: mutant III

FIG. 4 shows a cleavage map of recombinant plamid according to the present invention.

### BEST MODE

According to a first aspect, the present invention provides a DNA polymerase consisting essentially of amino acids sequence from 91 to 106 and from 205 to 220 of SEQ ID NO: 1. Specifically, said DNA polymerase consisting essentially of amino acids sequence from 91 to 315 of SEQ ID NO: 1. More specifically, said DNA polymerase consisting of amino acids sequence of SEQ ID NO: 1. Also, the present invention provides a DNA polymerase gene encoding amino acids sequence of SEQ ID NO: 1. Also, the present invention provides a recombinant vector containing said DNA polymerase gene and a host cell transformed with said recombinant vector.

According to a second aspect, the present invention provides a DNA polymerase consisting essentially of amino acids sequence from 91 to 106, from 133 to 148, from 205 to 220 and from 300 to 315 of SEQ ID NO: 2. Specifically, said DNA polymerase consisting essentially of amino acids sequence from 91 to 315 of SEQ ID NO: 2. More specifically, said DNA polymerase consisting of amino acids sequence of SEQ ID NO: 2. Also, the present invention provides a DNA polymerase gene encoding amino acids sequence of SEQ ID NO: 2. Also, the present invention provides a recombinant vector containing said DNA polymerase gene and a host cell transformed with said recombinant vector.

According to a third aspect, the present invention provides a DNA polymerase consisting essentially of amino acids sequence from 91 to 106, from 107 to 122, from 133 to 148, from 205 to 220 and from 300 to 315 of SEQ ID NO: 3. Specifically, said DNA polymerase consisting essentially of amino acids sequence from 91 to 315 of SEQ ID NO: 3. More specifically, said DNA polymerase consisting of amino acids sequence of SEQ ID NO: 3. Also, the present invention provides a DNA polymerase gene encoding amino acids sequence of SEQ ID NO: 3. Also, the present invention provides a recombinant vector containing said DNA polymerase gene and a host cell transformed with said recombinant vector.

According to a fourth aspect, the present invention provides expression plasmids comprising one of a DNA polymerase gene selected from the group of SEQ ID NO: 1, 2 and 3, and a method for producing a DNA polymerase using said transformed host cells. More specifically, the present invention provides a method for producing a DNA polymerase, comprising culturing cells transformed with an expression plasmid comprising a mutant DNA polymerase gene inducing expression of the recombinant protein according to the present invention and purifying the mutant DNA polymerase.

As used herein, the term “DNA polymerase” refers to an enzyme that synthesizes DNA in the 5′->3′ direction from deoxynucleotide triphosphate by using a complementary template DNA strand and a primer by successively adding nucleotide to a free 3′-hydroxyl group. The template strand determines the sequence of the added nucleotide by Watson-Crick base pairing.

As used herein, the term “functional equivalent” is intended to include amino acid sequence variants having amino acid substitutions in some or all of a DNA polymerase, or amino acid additions or deletions in some of the DNA polymerase. The amino acid substitutions are preferably conservative substitutions. Examples of the conservative substitutions of naturally occurring amino acids as follows; aliphatic amino acids (Gly, Ala, and Pro), hydrophobic amino acids (Ile, Leu, and Val), aromatic amino acids (Phe, Tyr, and Trp), acidic amino acids (Asp, and Glu), basic amino acids (His, Lys, Arg, Gln, and Asn), and sulfur-containing amino acids (Cys, and Met). It is preferable that the deletions of amino acids in DNA polymerase are located in a region where it is not directly involved in the activity of the DNA polymerase.

The present invention provides a DNA fragment encoding the mutant DNA polymerase. As used herein, the term “DNA fragment” includes sequences encoding the DNA polymerase of SEQ ID NO: 1 to 3, their functional equivalents and functional derivatives. Furthermore, the present invention provides various recombination vectors containing said DNA fragment, for example a plasmid, cosmid, phasimid, phase and virus. Preparation methods of said recombination vector are well known in the art.

As used herein, the term “vector” means a nucleic acid molecule that can carry another nucleic acid bound thereto. As used herein, the term “expression vector” is intended to include a plasmid, cosmid or phage, which can synthesize a protein encoded by a recombinant gene carried by said vector. A preferred vector is a vector that can self-replicate and express a nucleic acid bound thereto.

As used herein, the term “transformation” means that foreign DNA or RNA is absorbed into cells to change the genotype of the cells.

Cells suitable for transformation include prokaryotic, fungal, plant and animal cells, but are not limited thereto. Most preferably, E. coli cells are used.

Hereinafter, the present invention will be described in further detail with reference to examples. It is to be understood, however, that these examples are for illustrative purposes only and are not to be construed to limit the scope of the present invention.

### MODE FOR INVENTION

### Reference Example 1

### Cloning and Primary Sequence Analysis of Wild Type DNA Polymerase TNA1_pol Gene

Thermococcus sp. NA1 was isolated from deep-sea hydrothermal vent area at the PACMANUS field (3° 14′ S, and 151° 42′ E) in Papua New Guinea. An YPS medium was used to culture Thermococcus sp. NA1 for DNA manipulation, and the culture and maintenance of Thermococcus sp. NA1 were conducted according to standard methods. To prepare a Thermococcus sp. NA1 seed culture, an YPS medium in a 25-ml serum bottle was inoculated with a single colony formed on a phytagel plate, and cultured at 90° C. for 20 hours. The seed culture was used to inoculate 700 ml of an YPS medium in an anaerobic jar, and was cultured at 90° C. for 20 hours.

### Reference Example 2

### Preparation of Wild Type DNA Polymerase TNA1_pol Gene

E. coli DH5α was used for plasmid propagation including DNA polymerase TNA1_pol gene isolated from Thermococcus sp. and nucleotide sequence analysis. E. coli BL21-Codonplus(DE3)-RIL cells (Stratagene, La Jolla, Calif.) and plasmid pET-24a(+) (Novagen, Madison, Wis.) were used for gene expression. The E. coli strain was cultured in a Luria-Bertani medium at 37° C., and kanamycin was added to the medium to a final concentration of 50 μg/ml.

Also, DNA manipulation was conducted according to a standard method as described by Sambrook and Russell. The genomic DNA of Thermococcus sp. NA1 was isolated according to a standard method. Restriction enzymes and other modifying enzymes were purchased from Promega (Madison, Wis.). The preparation of a small scale of plasmid DNA from the E. coli cells was performed using the plasmid mini-kit (Qiagen, Hilden, Germany). The sequence analysis of DNA was performed with an automated sequencer (ABI3100) using the BigDye terminator kit (PE Applied Biosystems, Foster City, Calif.).

Through the genomic sequence analysis, an open reading frame (3,927 bp) encoding a protein consisting of 1,308 amino acids was found, and it showed a very high similarity to the family B DNA polymerases. The molecular mass of a protein derived from the deduced amino acid sequence was 151.9 kDa, which was much larger than the size predicted for the average molecular mass thermostable DNA polymerases. The sequence analysis showed that the DNA polymerase gene contained a putative 3′-5′ exonuclease domain, an α-like DNA polymerase domain, and a 1605-bp (535 amino acids) in-frame intervening sequence in the middle of a region (Pol III) conserved between the α-like DNA polymerases of eukaryotes and archaeal (Pol III). Also, the deduced amino acid sequence of the intein of the polymerase was highly similar to the intein of the polymerase of other archaeal, and exhibited a identity of 81.0% to a pol_{—}1 intein 1 (derived from a DNA polymerase of Thermococcus sp. strain GE8; 537 amino acids; AJ25033), a identity of 69.0% to IVS-B (derived from KOD DNA polymerase; 537 amino acids; D29671) and a homology of 67.0% to an intein (derived from deep vent DNA polymerase; 537 amino acids; U00707).

Also, the splicing site of the intein could be predicted by sequence analysis, because Cys or Per was well conserved in the N-terminus of the intein, and His-Asn-Cys/Ser/Thr was well conserved in the C-terminal splice junction. Thus, a mature polymerase gene (TNA1_pol) containing no intein could be predicted, and it would be a 2,322-bp sequence encoding a protein consisting of 773 amino acid residues. The deduced sequence of TNA1_pol was compared with those of other DNA polymerases. In pairwise alignment, the deduced amino acid sequence of the mature TNA1_pol gene showed a identity of 91.0% to KOD DNA polymerase (gi: 52696275), a identity of 82.0% to deep vent DNA polymerase (gi: 436495), and a identity of 79.0% to pfu DNA polymerase (gi: 18892147). To examine the performance of TNA1_pol in PCR amplification, TNA1_pol DNA was constructed by removing the intein from the full-length polymerase as described above.

The mature DNA polymerase containing no intein was constructed in the following manner. Using primers designed to contain overlapping sequences, each of the TNA1-pol N-terminal and C-terminal portion was amplified. Then, the full length of a TNA1_pol gene flanked by NdeI and XhoI sites was amplified by PCR using two primers and a mixture of said partially PCR amplified N-terminal and C-terminal fragments as a template. The amplified fragment was digested with NdeI and XhoI, and ligated with pET-24a(+) digested with NdeI/XhoI. The ligate was transformed into E. coli DH5α. Candidates having a correct construct were selected by restriction enzyme digestion, and were confirmed to have a mature DNA polymerase by analyzing the DNA sequence of the clones.

### Example 1

### Construction of Mutant NA1 DNA Polymerase by Site-Specific Mutagenesis

To prepare mutant DNA polymerase NA1, site-specific mutagenesis were carried out according to the protocol using PCR with various synthetic primers corresponding to the specific site, respectively. Primers for the mutation and prepared mutant DNA polymerases were listed in Table 1.

TABLE 1
PCR Primer sequences for site-specific
mutagenesis of the DNA polymerase

| object | Forward primer | Reverse priemr |
|---|---|---|
| DNA | CACCCGCAGGACCAACCCGCAATCCGC | GCGGATTGCGGGTTGGTCCTGCGGGTGC |
| polymerase | GACAAGATAAGG | TCGAAGTAG |
| of SEQ ID | (SEQ ID NO: 4) | (SEQ ID NO: 5) |
| NO: 1 | CTCATTACCTACGACGGCGACAACTTT | AAAGTTGTCGCCGTCGTAGGTAATGAGA |
| | GACTTTGCTTAC | ACATCAGGATC |
| | (SEQ ID NO: 6) | (SEQ ID NO: 7) |
| DNA | CACCCGCAGGACCAGCCCGCAATCCGC | GCGGATTGCGGGCTGGTCCTGCGGGTGC |
| polymerase | GACAAGATAAGG | TCGAAGTAG |
| of SEQ ID | (SEQ ID NO: 8) | (SEQ ID NO: 9) |
| NO: 2 | ATGCTCGCCTTTGCCATCGAGACGCTC | GAGCGTCTCGATGGCAAAGGCGAGCATC |
| | TACCACGAGGGC | TTCAGTTCTTC |
| | (SEQ ID NO: 10) | (SEQ ID NO: 11) |
| | CTCATTACCTACGACGGCGACAACTTT | AAAGTTGTCGCCGTCGTAGGTAATGAGA |
| | GACTTTGCTTAC | ACATCAGGATC |
| | (SEQ ID NO: 6) | (SEQ ID NO: 7) |
| | CGCGTTGCGCGCTTCTCTATGGAAGAT | ATCTTCCATAGAGAAGCGCGCAACGCGC |
| | GCAAAGGCAACC | TCAAGCCCCTC |
| | (SEQ ID NO: 12) | (SEQ ID NO: 13) |
| | CACCCGCAGGACCAGCCCGCAATCCGC | GCGGATTGCGGGCTGGTCCTGCGGGTGC |
| | GACAAGATAAGG | TCGAAGTAG |
| | (SEQ ID NO: 8) | (SEQ ID NO: 9) |
| DNA | ATGCTCGCCTTTGCCATCGAGACGCTC | GAGCGTCTCGATGGCAAAGGCGAGCATC |
| polymerase | TACCACGAGGGC | TTCAGTTCTTC |
| of SEQ ID | (SEQ ID NO: 10) | (SEQ ID NO: 11) |
| NO: 3 | CTCATTACCTACGACGGCGACAACTTT | AAAGTTGTCGCCGTCGTAGGTAATGAGA |
| | GACTTTGCTTAC | ACATCAGGATC |
| | (SEQ ID NO: 6) | (SEQ ID NO: 7) |
| | CGCGTTGCGCGCTTCTCTATGGAAGAT | ATCTTCCATAGAGAAGCGCGCAACGCGC |
| | GCAAAGGCAACC | TCAAGCCCCTC |
| | (SEQ ID NO: 12) | (SEQ ID NO: 13) |
| | CGACATACCCCGCGCCAAGCGCTACCT | GCGCTTGGCGCGGGGTATGTCGTACTCG |
| | C | (SEQ ID NO: 15) |
| | (SEQ ID NO: 14) | |

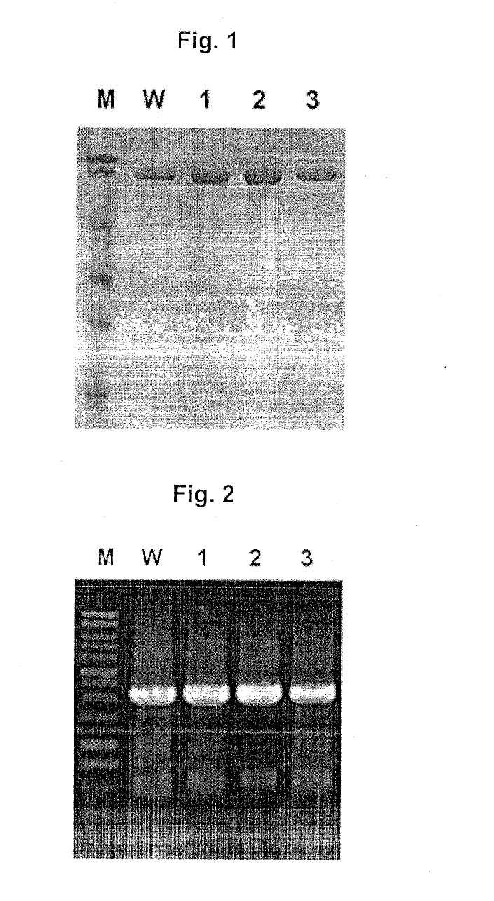

FIG. 1 shows the results of SDS-PAGE analysis of mutant DNA polymerases. M: a standard sample, W: wild type, 1: mutant I, 2: mutant II, 3: mutant III

The PCR reaction was performed in the following conditions: a single denaturation step of 5 min at 95° C., and then 15 cycles with a temperature profile of 15 sec at 95° C., 1 sec at 55° C. and 20 sec at 72° C., followed by final extension for 7 min at 72° C.

FIG. 2 shows the results of PCR analysis using mutant DNA polymerases, respectively.

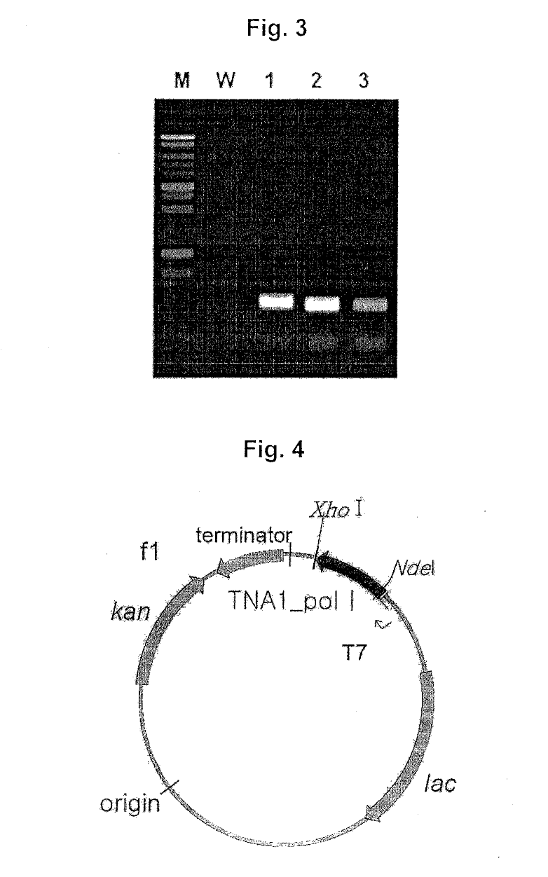

FIG. 3 shows the results of PCR analysis using mutant DNA polymerases and primer with inosine, respectively.

### Example 2

### Expression and Purification of the Mutant NA1 DNA Polymerases

The pET system having a very strong, stringent T7/lac promoter, is one of the most powerful systems developed for the cloning and expression of a heterologus proteins in E. coli. The mutant NA1 polymerase gene purified from example 1 was inserted into the NdeI and XhoI sites of plasmid vector pET-24a(+) in order to facilitate the over-expression and the His-tagged purification of the recombinant protein (FIG. 4). The mutant NA1 polymerase was expressed in a soluble form in the cytosol of E. coli BL21-codonPlus(DE3)-RIL transformed with said recombinant expression plasmid.

Specifically, overexpression of the mutant NA1 polymerase was induced by adding isopropyl-β-D-thiogalactopyranoside (IPTG) in the mid-exponential growth stage, followed by constant-temperature incubation at 37° C. for 3 hours. The cells were harvested by centrifugation (at 4° C. and 6,000× g for 20 minutes), and re-suspended in a 50 mM Tris-HCl buffer (pH 8.0) containing 0.1M KCl and 10% glycerol. The cells were ultrasonically disrupted, and isolated by centrifugation (at 4° C. and 20,000× g for 30 minutes), and a crude enzyme sample was thermally treated at 80° C. for 20 minutes. The resulting supernatant was treated in a column of TALON™ metal affinity resin (BD Bioscience Clontech, Palo Alto, Calif.), and washed with 10 mM imidazole (Sigma, St. Louis, Mo.) in a 50 mM Tris-HCl buffer (pH 8.0) containing 0.1 M KCl and 10% glycerol, and mutant NA1 polymerase was eluted with 300 mM imidazole in buffer. The pooled fractions were dialyzed into a storage buffer containing 50 mM Tris-HCl (pH 7.5), 1 mM DTT, 1 mM EDTA and 10% glycerol. The concentrations of proteins were determined by the colorimetric assay of Bradford. The purification degrees of the proteins were examined by sodium dodecyl sulfate-polyacrylamide gel electrophoresis (SDS-PAGE) analysis according to a standard method (FIG. 1)

The thermal treatment conducted at 80° C. for 20 minutes could eliminate effectively several E. coli proteins. However, some E. coli proteins remained in a stable form after the thermal treatment. The soluble supernatant of the heat-treated pool was chromatographied on a column of TALON™ metal affinity resin. The specific activity of the purified protein was 231.33 units/mg, and the purification yield was 26.155%. SDS-PAGE analysis revealed a major protein hand with a molecular mass of 80 kDa. The purified proteins remained soluble in repeated freezing and thawing cycles.

TABLE 2
Isolation of TNA1_pol from E. coli

| | Total | Total | Specific | |
|---|---|---|---|---|
| Purification | protein | activity | activity | Yield |
| step | (mg) | (U) | (U/mg) | (%) |
| Crude extract | 46.6 | 2915.26 | 62.62 | 100 |
| Thermal | 29.7 | 2518.62 | 127.85 | 36.31 |
| denaturation | | | | |
| His-tagged | 3.3 | 763.37 | 231.33 | 26.15 |
| affinity column | | | | |

### Example 3

### PCR Analysis Using Mutant NA1 DNA Polymerases

The major application of thermostable mutant DNA polymerases is the in vitro amplification of DNA fragments. To test the performance of recombinant polymerases for in vitro amplification, said enzymes was applied to PCR reaction. 2.5 U of each of various DNA polymerases was added to 50 μl of a reaction mixture containing 50 ng of genomic DNA from Thermococcus sp. NA1 as a template, 10 pmole of each primer, 200 μM dNTP, and PCR reaction buffer. To amplify a 2-kb fragment from the genomic DNA of Thermococcus sp. NA1, primers were designed. PCR buffer supplied by the manufacturer was used in the amplification of the commercial polymerases. Also, for the PCR amplification of said polymerase, a buffer consisting of 20 mM Tris-HCl (pH 8.5), 30 mM (NH₄)₂SO₄, 60 mM KCl and 1 mM MgCl₂ was used. The PCR reaction was performed in the following conditions: a single denaturation step at 95° C., and then 30 cycles with a temperature profile of 1 min at 94° C., 1 min at 55° C. and 2 min at 72° C., followed by final extension for 7 min at 72° C. The PCR products were analyzed in 0.8% agarsose gel electrophoresis. To test the performance of recombinant polymerases on the amplification of long-chain DNA, PCR reaction was carried out in 50 μl of a reaction mixture containing 50 ng of genomic DNA from Thermococcus sp. NA1 as a template, 200 μM dNTP, and PCR reaction buffer. PCR was performed using mutant DNA polymerses according to the present invention and primers with inosine. As the result, DNA polymerases according to the present invention were amplified high-efficiently more than that of wild type (FIG. 3).

### INDUSTRIAL APPLICABILITY

As described above, the present invention relates to DNA polymerases which are produced by site-specific mutagenesis from the isolated Thermococcus sp NA1. strain, their amino acid sequences, genes encoding said mutant DNA polymerases, their sequences, preparation methods thereof and use of PCR using thereof. As mutant DNA polymerases according to the present invention has the changed function of exonuclease and inosine sensing simultaneously, the present invention is broadly applicable for PCR using primers with inosine in various molecular genetic technology.

1. A DNA polymerase consisting essentially of amino acids sequence from 91 to 106 and from 205 to 220 of SEQ ID NO: 1.

2. The DNA polymerase according to claim 1, which consists essentially of amino acids sequence from 91 to 315 of SEQ ID NO: 1.

3. The DNA polymerase according to claim 1, which consists of amino acids sequence of SEQ ID NO: 1.

4. A DNA polymerase gene encoding amino acids sequence of SEQ ID NO: 1.

5. A recombinant vector comprising the DNA polymerase gene of claim 4.

6. A host cell transformed with the recombinant vector of claim 5.

7. A DNA polymerase consisting essentially of amino acids sequence from 91 to 106, from 133 to 148, from 205 to 220 and from 300 to 315 of SEQ ID NO: 2.

8. The DNA polymerase according to claim 7, which consists essentially of amino acids sequence from 91 to 315 of SEQ ID NO: 2.

9. The DNA polymerase according to claim 7, which consists of amino acids sequence of SEQ ID NO: 2.

10. A DNA polymerase gene encoding amino acids sequence of SEQ ID NO: 2.

11. A recombinant vector comprising the DNA polymerase gene of claim 10.

12. A host cell transformed with the recombinant vector of claim 11.

13. A DNA polymerase consisting essentially of amino acids sequence from 91 to 106, from 107 to 122, from 133 to 148, from 205 to 220 and from 300 to 315 of SEQ ID NO: 3.

14. The DNA polymerase according to claim 13, which consists essentially of amino acids sequence from 91 to 315 of SEQ ID NO: 3.

15. The DNA polymerase according to claim 13, which consists of amino acids sequence of SEQ ID NO: 3.

16. A DNA polymerase gene encoding amino acids sequence of SEQ ID NO: 3.

17. A recombinant vector comprising the DNA polymerase gene of claim 16.

18. A host cell transformed with the recombinant vector of claim 17.

19. (canceled)

20. A method for producing a DNA polymerase, comprising the steps: (a) culturing the host cell of claim 6; (b) inducing expression of the recombinant protein; and (c) purifying the DNA polymerase.

21. A method for producing a DNA polymerase, comprising the steps: (a) culturing the host cell of claim 12; (b) inducing expression of the recombinant protein; and (c) purifying the DNA polymerase.

22. A method for producing a DNA polymerase, comprising the steps: (a) culturing the host cell of claim 18; (b) inducing expression of the recombinant protein; and (c) purifying the DNA polymerase.


SEQUENCE LISTING (15 sequences)

SEQ ID NO: 1 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDQPAIRDKIRAHPGVIDIYEYDIPFAKRYLIDKGLVPMEGDEELKMLAFDIETLYHEGEEFGTGPILMISYADENEARVITWKKIDLPYVDVVSTEKEMIKRFLRVVKEKDPDVLITYDGDNFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARYSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 2 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDEPAIRDKIRAHPGVIDIYEYDIPFAKRYLIDKGLVPMEGDEELKMLAFAIETLYHEGEEFGTGPILMISYADENEARVITWKKIDLPYVDVVSTEKEMIKRFLRVVKEKDPDVLITYDGDNFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARFSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 3 - protein, 773 aa, Thermococcus sp. NA1
MILDVDYITEDGKPVIRIFKKEKGEFKIEYDRDFEPYIYALLKDDSAIEEVKKITAERHGKVVKVKRAEKVNKKFLGRPVEVWKLYFEHPQDEPAIRDKIRAHPGVIDIYEYDIPRAKRYLIDKGLVPMEGDEELKMLAFAIETLYHEGEEFGTGPILMISYADENEARVITWKKIDLPYVDVVSTEKEMIKRFLRVVKEKDPDVLITYDGDNFDFAYLKKRCEKLGISFTLGRDGSEPKIHRMGDRFAVEVKGRIHFDLYPVIRRTINLPTYTLEVVYEAVFGKPKEKVYAEEITLAWESGEGLERVARFSMEDAKATYELGREFFPMEAQLSRLIGQSLWDVSRSSTGNLVEWFLLRKAYERNELAPNKPDEGELARRRNSYAGGYVKEPERGLWDNIVYLDFRSLYPSIIITHNVSPDTLNREGCKEYDVAPQVGHKFCKDFPGFIPSLLGNLLEERQKIKRKMKATIDPLEKKLLDYRQRAIKILANSYYGYYGYPRARWYCKECAESVTAWGREYIEMTIREIEEKYGFKVLYADTDGFYATIPGADAETVKKKAKEFLKYINAKLPGLLELEYEGFYKRGFFVTKKKYAVIDEEGKIVTRGLEIVRRDWSDIAKETQARVLEALLKDGNVEKAVKIVKEITEKLSKYEIPPEKLVIHEQITRELKDYKATGPHVAIAKRLAARGIKVRPGTIISYIVLKGSGRIGDRAIPFDEFDPTKHKYDADYYIENQVLPAVMRILEAFGYKKEDLRYQKTRQVGLGAWLKPKK

SEQ ID NO: 4 - DNA, 39 bp, Artificial Sequence
  93Q forward primer
cacccgcaggaccaacccgcaatccgcgacaagataagg

SEQ ID NO: 5 - DNA, 37 bp, Artificial Sequence
  93Q Reverse primer
gcggattgcgggttggtcctgcgggtgctcgaagtag

SEQ ID NO: 6 - DNA, 39 bp, Artificial Sequence
  210D Forward primer
ctcattacctacgacggcgacaactttgactttgcttac

SEQ ID NO: 7 - DNA, 39 bp, Artificial Sequence
  210D Reverse primer
aaagttgtcgccgtcgtaggtaatgagaacatcaggatc

SEQ ID NO: 8 - DNA, 39 bp, Artificial Sequence
  93E Forward primer
cacccgcaggaccagcccgcaatccgcgacaagataagg

SEQ ID NO: 9 - DNA, 37 bp, Artificial Sequence
  93E Reverse primer
gcggattgcgggctggtcctgcgggtgctcgaagtag

SEQ ID NO: 10 - DNA, 39 bp, Artificial Sequence
  141A Forward primer
atgctcgcctttgccatcgagacgctctaccacgagggc

SEQ ID NO: 11 - DNA, 39 bp, Artificial Sequence
  141A Reverse primer
gagcgtctcgatggcaaaggcgagcatcttcagttcttc

SEQ ID NO: 12 - DNA, 39 bp, Artificial Sequence
  311F Forward primer
cgcgttgcgcgcttctctatggaagatgcaaaggcaacc

SEQ ID NO: 13 - DNA, 39 bp, Artificial Sequence
  311F Reverse primer
atcttccatagagaagcgcgcaacgcgctcaagcccctc

SEQ ID NO: 14 - DNA, 28 bp, Artificial Sequence
  116R Forward primer
cgacataccccgcgccaagcgctacctc

SEQ ID NO: 15 - DNA, 28 bp, Artificial Sequence
  116R Reverse primer
gcgcttggcgcggggtatgtcgtactcg